(19) United States
(12) Patent Application Publication
(10) Pub. No.: US 2025/0025133 A1
(43) Pub. Date: Jan. 23, 2025

(54) ULTRASONIC IMAGING METHOD AND DEVICE

(71) Applicant: SHENZHEN MINDRAY BIO-MEDICAL ELECTRONICS CO., LTD., Shenzhen (CN)

(72) Inventors: Xiao HAN, Shenzhen (CN); Guohao DONG, Shenzhen (CN); Yaoxian ZOU, Shenzhen (CN); Muqing LIN, Shenzhen (CN); Tao JIN, Shenzhen (CN)

(21) Appl. No.: 18/902,601

(22) Filed: Sep. 30, 2024

Related U.S. Application Data
(63) Continuation of application No. 17/359,615, filed on Jun. 27, 2021.
Application No. 17/359,615 is a continuation of application No. PCT/CN2018/125832, filed on Dec. 29, 2018.

(51) Int. Cl. A61B 8/08; A61B 8/00; G01S 7/52; G01S 15/89; G06T 7/11; G06T 7/13; G06T 7/73

(52) U.S. Cl. CPC A61B 8/483; A61B 8/085; A61B 8/466; A61B 8/58; G01S 7/52053; G01S 15/8993; G06T 7/11; G06T 7/13; G06T 7/74; G06T 2207/10136; G06T 2207/20036; G06T 2207/20081; G06T 2207/30004

(57) ABSTRACT

An ultrasonic imaging method and device and a computer-readable storage medium are provided. The ultrasonic imaging method includes: ultrasonic waves are transmitted to a uterine region of an object to be detected; ultrasonic echoes are received based on the ultrasonic waves reflected from the uterine region of said object, and ultrasonic echo signals are acquired on the basis of the ultrasonic echoes; the ultrasonic echo signals are processed to obtain three-dimensional data of the uterine region of said object; the endometrium is identified from the three-dimensional data of the uterine region according to endometrium image characteristics of the uterine region to obtain position information of the identified endometrium; according to the position information of the identified endometrium, the cross section of the identified endometrium is imaged on the basis of the three-dimensional data to obtain an cross-sectional endometrium image; and the cross-sectional endometrium image is displayed.

### CROSS-REFERENCE TO RELATED APPLICATIONS

This application is a continuation application of U.S. patent application Ser. No. 17/359,615, filed on Jun. 27, 2021, which is a continuation of International Patent Application No. PCT/CN2018/125832 filed on Dec. 29, 2018, the contents of both of which are incorporated herein by reference in their entirety.

### TECHNICAL FIELD

Embodiments of the disclosure relate to the technical field of ultrasound imaging, and in particular to an ultrasound imaging method and device, and a computer-readable storage medium.

### BACKGROUND

In modern medical imaging examination, an ultrasound technology has become the most widely and most frequently applied and fastest popularized examination means because of its high reliability, quickness and convenience, real-time imaging, repeatable examination, etc. In particular, the development of artificial intelligence-assisted technologies has further promoted the application of the ultrasound technology in clinical diagnosis and treatment.

Gynecologic ultrasonography is one of the relatively important and widely applied fields in ultrasound diagnosis. Ultrasonography of the uterus and uterine appendages can provide important guidance for the diagnosis and treatment of many gynecological diseases. Three-dimensional ultrasound can present a coronal section ultrasonogram of the uterus, which clearly shows whether the endometrium has lesions and whether its morphology is complete. Therefore, it is of great significance to use the three-dimensional ultrasound technology to implement the diagnosis of uterus-related gynecological diseases.

Although the three-dimensional ultrasound technology has the above advantages, for reasons such as easily confused coordinate axes of a three-dimensional volume image, various orientation changes of the uterus, and a relatively abstract three-dimensional space, a doctor may need to rotate the three-dimensional volume image repeatedly to find section by section for a standard section of the endometrium when manually searching for a uterine part and determining a standard section image of the endometrium. This manual locating process is not only time-consuming and labor-consuming, but also has limited intelligence and accuracy of imaging.

### SUMMARY

An embodiment of the disclosure provides an ultrasound imaging method, the method comprising:
transmitting ultrasonic waves to a uterine region of an object to be detected for volume scanning;
receiving ultrasonic echoes returned from the uterine region of the object to be detected, and acquiring ultrasonic echo signals based on the ultrasonic echoes;
processing the ultrasonic echo signals to obtain three-dimensional volume data of the uterine region of the object to be detected;
identifying an endometrium from the three-dimensional volume data of the uterine region according to an image characteristic of the endometrium in the uterine region, so as to obtain position information of the endometrium;
performing imaging on the endometrium based on the three-dimensional volume data according to the position information of the endometrium, so as to obtain an image of the endometrium; and
displaying the image of the endometrium.

An embodiment of the disclosure further provides an ultrasound imaging method, the method comprising:
performing ultrasonic volume scanning on an object to be detected, so as to obtain three-dimensional volume data of the object to be detected;
identifying a region of interest from the three-dimensional volume data of the object to be detected according to an image characteristic of the region of interest in the object to be detected, so as to obtain position information of the region of interest;
processing the three-dimensional volume data according to the position information of the region of interest, so as to obtain an image of the region of interest; and
displaying the image of the region of interest.

An embodiment of the disclosure provides an ultrasound imaging device, the ultrasound imaging device comprising:
a probe;
a transmitting circuit configured to excite the probe to transmit ultrasonic waves to an object to be detected for volume scanning;
a transmitting/receiving selection switch;
a receiving circuit configured to receive, by the probe, ultrasonic echoes returned from the object to be detected, so as to obtain ultrasonic echo signals/data;
a beam synthesis circuit configured to perform beam synthesis processing on the ultrasonic echo signals/data to obtain ultrasonic echo signals/data, which have been subjected to beam synthesis;
a processor configured to: process the ultrasonic echo signals, which have been subjected to beam synthesis, so as to obtain three-dimensional volume data of the uterine region of the object to be detected; identify an endometrium from the three-dimensional volume data of the uterine region according to an image characteristic of the endometrium in the uterine region, so as to obtain position information of the endometrium; and perform imaging on the endometrium based on the three-dimensional volume data according to the position information of the endometrium, so as to obtain an image of the endometrium; and
a display configured to display the image of the endometrium.

An embodiment of the disclosure provides a computer-readable storage medium storing an ultrasound imaging program, wherein the ultrasound imaging program may be executed by a processor to implement the above ultrasound imaging method.

Embodiments of the disclosure provide an ultrasound imaging method and device, and a computer-readable storage medium. Through the above technical implementation solutions, the ultrasound imaging device may automatically obtain position information of endometrium according to an image characteristic of the endometrium, thereby omitting cumbersome operations of continuously and manually locating the endometrium by a user, facilitating quick identification of the endometrium by the user, and improving the overall working efficiency.

The ultrasound imaging device may further perform automatic imaging according to the position information of the endometrium to obtain an image of the endometrium, and since the automatically identified position of the endometrium is accurate, the accuracy of subsequent ultrasound imaging is improved, and the automatic imaging may also improve the intelligence of the imaging of an ultrasound image.

### BRIEF DESCRIPTION OF THE DRAWINGS

FIG. 1 is a schematic structural block diagram of an ultrasound imaging device according to an embodiment of the disclosure;

FIG. 2 is a flow chart 1 of an ultrasound imaging method according to an embodiment of the disclosure;

FIG. 3 is a block diagram 1 of an exemplary ultrasound imaging procedure according to an embodiment of the disclosure;

FIG. 4 is a schematic diagram of an exemplary VOI box according to an embodiment of the disclosure;

FIG. 5 is an exemplary result of VR imaging according to an embodiment of the disclosure;

FIG. 6 is a schematic diagram of an exemplary CMPR imaging process according to an embodiment of the disclosure;

FIG. 7 is an exemplary result of CMPR imaging according to an embodiment of the disclosure;

FIG. 8 is a block diagram 2 of an exemplary ultrasound imaging procedure according to an embodiment of the disclosure;

FIG. 9 is a flow chart 2 of an ultrasound imaging method according to an embodiment of the disclosure; and

FIG. 10 is a schematic diagram of a transverse image of endometrium according to an embodiment of the disclosure.

### DESCRIPTION OF THE PREFERRED EMBODIMENTS

In order to understand the characteristics and technical contents of embodiments of the disclosure in more detail, the implementation of the embodiments of the disclosure will be described below in detail with reference to the accompanying drawings, which are for reference and illustration only, and are not intended to limit the embodiments of the disclosure.

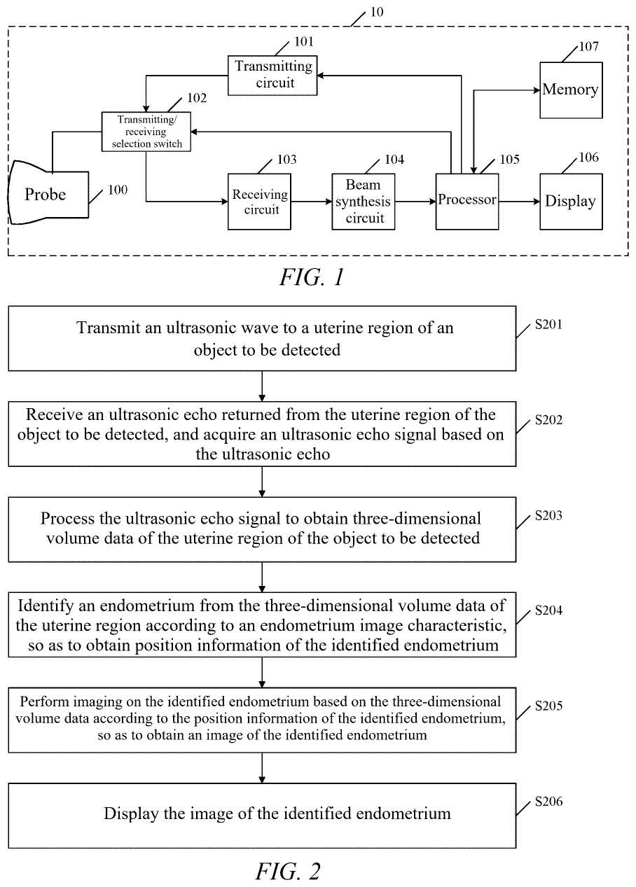

FIG. 1 is a schematic structural block diagram of an ultrasound imaging device in an embodiment of the disclosure. An ultrasound imaging device 10 may comprise an ultrasonic probe 100, a transmitting circuit 101, a transmitting/receiving selection switch 102, a receiving circuit 103, a beam synthesis circuit 104, a processor 105, and a display 106. The transmitting circuit 101 can excite the probe 100 to transmit ultrasonic waves to a target tissue; the receiving circuit 103 can receive, by the probe 100, ultrasonic echoes reflected from an object to be detected, so as to obtain ultrasonic echo signals/data; and the ultrasonic echo signals/data are subjected to beam synthesis processing by the beam synthesis circuit 104 and are then sent to the processor 105. The processor 105 processes the ultrasonic echo signals/data to obtain an ultrasound image of the object to be detected. The ultrasound image obtained by the processor 105 may be stored in a memory 107. These ultrasound images may be displayed on the display 106.

In one embodiment of the disclosure, the display 106 of the above ultrasound imaging device 10 may be a touch display screen, a liquid crystal display screen, etc., or may be an independent display device, such as a liquid crystal display or a television, independent of the ultrasound imaging device 10, or may be a display screen on an electronic device, such as a mobile phone and a tablet computer.

In practical applications, the processor 105 may be at least one of an application specific integrated circuit (ASIC), a digital signal processor (DSP), a digital signal processing device (DSPD), a programmable logic device (PLD), a field programmable gate array (FPGA), a central processing unit (CPU), a controller, a microcontroller, and a microprocessor, such that the processor 105 can perform the corresponding steps of the ultrasound imaging methods in the embodiments of the disclosure.

The memory 107 may be a volatile memory, such as a random access memory (RAM), or a non-volatile memory, such as a read-only memory (ROM), a flash memory, a hard disk drive (HDD) or a solid-state drive (SSD), or a combination of the above types of memories, and provide instructions and data to the processor.

The technical solution of the disclosure will be described below in detail based on the above ultrasound imaging device 10.

An embodiment of the disclosure provides an ultrasound imaging method, and as shown in FIG. 2, the method may comprise the steps as follows.

S101. An ultrasonic wave is transmitted to a uterine region of an object to be detected for volume scanning.

In the embodiment of the disclosure, the ultrasound imaging device may transmit, by a probe, the ultrasonic waves to the uterine region of the object to be detected, so as to implement ultrasonic scanning and inspection of the uterine region, and same is used in a scenario in which the uterine region is to be detected.

It should be noted that the object to be detected may be an object comprising a uterine region, such as a human organ or a human tissue structure, wherein the uterine region herein is a region comprising all or part of the uterus, or comprising all or part of the uterus and uterine appendages.

In the embodiment of the disclosure, the ultrasound imaging device can identify a key anatomical structure of the uterine region, and the uterine region is represented by the position of the key anatomical structure. The key anatomical structure of the uterine region herein may be endometrium. Therefore, in the embodiment of the disclosure, the position of the endometrium is identified to represent an ultrasound image of the uterine region.

S102. An ultrasonic echo reflected from the uterine region of the object to be detected is received, and an ultrasonic echo signal is acquired based on the ultrasonic echo.

S103. The ultrasonic echo signal is processed to obtain three-dimensional volume data of the uterine region of the object to be detected.

The receiving circuit of the ultrasound imaging device can receive, by the probe, the ultrasonic echoes reflected from the uterine region of the object to be detected, so as to obtain the ultrasonic echo signals/data. The ultrasonic echo signals/data are subjected to beam synthesis processing by a beam synthesis circuit and are then sent to a processor. The processor of the ultrasound imaging device performs signal processing and three-dimensional reconstruction on the ultrasonic echo signals/data to obtain the three-dimensional volume data of the uterine region of the object to be detected.

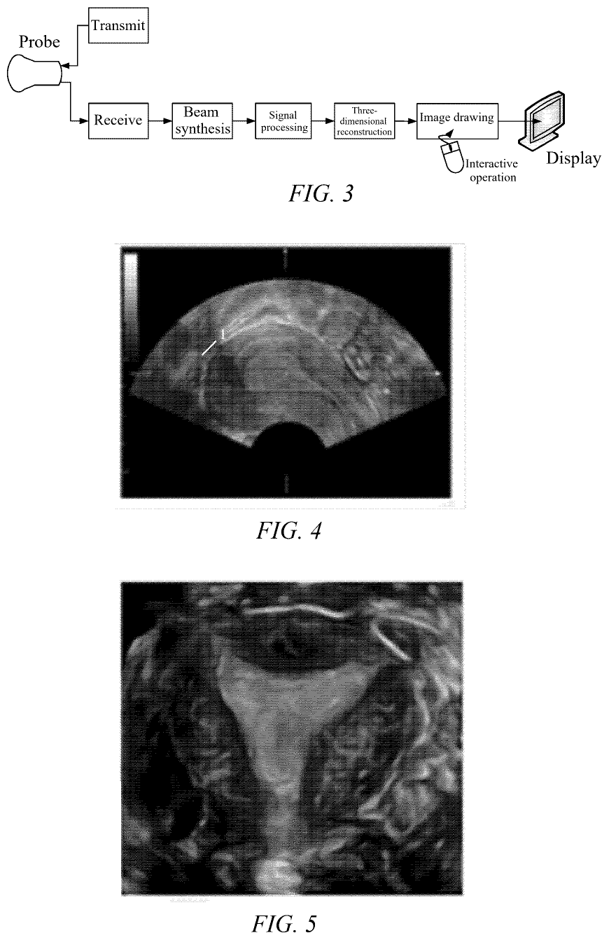

It should be noted that as shown in FIG. 3, the transmitting circuit sends a group of delayed focused pulses to the probe, and the probe transmits ultrasonic waves to a body tissue of the object to be detected, receives, after a certain delay, ultrasonic echoes that carry tissue information and are reflected from the body tissue of the object to be detected, and re-converts the ultrasonic echoes into electrical signals; the receiving circuit receives the electrical signals (ultrasonic echo signals), and sends the ultrasonic echo signals to a beam synthesis circuit; the ultrasonic echo signals undergo focusing delay, weighting and channel summation in the beam synthesis circuit, then signal processing in a signal processing module (i.e., a processor), and are then sent to a three-dimensional reconstruction module (i.e., the processor) for image drawing and rendering post-processing, such that a visual information ultrasound image is obtained and then transmitted to a display for display.

S104. An endometrium is identified from the three-dimensional volume data of the uterine region according to an endometrium image characteristic, so as to obtain position information of the identified endometrium.

In the embodiment of the disclosure, after obtaining the three-dimensional volume data of the uterine region of the object to be detected, the ultrasound imaging device can extract and compare characteristics of the three-dimensional volume data of the uterine region according to the endometrium image characteristics, so as to identify the endometrium, and thus obtain the position information of the identified endometrium.

It should be noted that before performing the three-dimensional reconstruction of the endometrium, the ultrasound imaging device needs to identify which anatomical structures are related to the endometrium to be determined. For example, in the volume data of the uterine region, echoes of the endometrium are significantly different from echoes of surrounding tissues, and as a woman's menstrual cycle changes, the morphology of the endometrium also shows periodic changes, with relatively obvious characteristics. Therefore, a section of the endometrium can be determined by using the endometrium as the key anatomical structure in the uterine region. In the embodiment of the disclosure, the detection of the key anatomical structure of the uterine region includes but is not limited the detection of the endometrium.

In some embodiments of the disclosure, the endometrium and a basal tissue of the uterus have different capabilities of reflecting ultrasonic waves, and grayscale characteristics of the correspondingly obtained ultrasonic echo signals are different. Therefore, the ultrasound imaging device can identify the endometrium from the three-dimensional volume data of the uterine region according to the difference between image characteristics of the endometrium and the basal tissue of the uterus in the uterine region. The ultrasound imaging device can determine the boundary between the endometrium and the basal tissue of the uterus according to the difference in grayscale values, so as to identify the endometrium from the three-dimensional volume data. In some embodiments of the disclosure, as a woman's menstrual cycle changes, the morphology of the endometrium also shows periodic changes. Therefore, the ultrasound imaging device can identify the endometrium from the three-dimensional volume data of the uterine region according to a periodically changing morphological characteristic of an endometrium in the uterine region, so as to obtain the position information of the identified endometrium. The ultrasound imaging device can identify the endometrium from the three-dimensional volume data of the uterine region based on the morphological characteristics of an endometrium in different periods of the menstrual cycle. There will be a detailed description below.

It should be noted that the method for identifying a key anatomical structure such as the endometrium may be performed manually or automatically. When manually acquiring an anatomical structure, a user can point and draw lines, by means of a keyboard, a mouse and other tools, on a specific anatomical structure in three-dimensional volume data through a certain workflow to inform the type and position of the key anatomical structure. In the embodiment of the disclosure, with the method for automatically identifying the endometrium, automatically identifying the endometrium refers to extracting characteristics from the three-dimensional volume data, and then automatically detecting the position of the endometrium in the three-dimensional volume data by using the characteristics.

In the embodiment of the disclosure, the method for automatically identifying a key anatomical structure is implemented in two cases: one is to directly determine a spatial position of the endometrium in the three-dimensional volume data; and the other is to detect the endometrium in the section of the three-dimensional volume data, and then determine the position of the endometrium in the three-dimensional volume data according to the position of the section position in the three-dimensional volume data and the position of the endometrium in the section. The position of the key anatomical structure such as the endometrium may be expressed by enclosing the anatomical position with a region of interest (ROI) box, or by accurately segmenting the boundary of the anatomical structure, or by using one or more points for auxiliary expression. There are many methods for automatically identifying such a key anatomical structure as the endometrium from the three-dimensional volume data, which are not limited in the embodiment of the disclosure.

Exemplarily, for the process of determining a spatial position of the endometrium from the three-dimensional volume data, so as to acquire the most standard section of the endometrium, it is possible to implement the detection of the endometrium based on the method for detecting characteristics such as grayscale and/or morphology, or to detect or accurately segment the endometrium in the three-dimensional volume data by using a machine learning or deep learning method, which is not limited in the embodiment of the disclosure.

In some embodiments of the disclosure, the following several implementations in which the ultrasound imaging device identifies the endometrium from the three-dimensional volume data of the uterine region according to an endometrium image characteristic in the uterine region so as to obtain the position information of the identified endometrium may be comprised, which are not limited in the embodiment of the disclosure.

In one embodiment of the disclosure, the ultrasound imaging device extracts a preset characteristic from the three-dimensional volume data of the uterine region to obtain at least one candidate region of interest; the ultrasound imaging device acquires three-dimensional template data of the uterine region where the endometrium has been recognized, and obtains a preset template region of the recognized endometrium according to the three-dimensional template data; and the ultrasound imaging device performs matching on the at least one candidate region of interest and the preset template region to identify a candidate region of interest with highest matching degree as a target region of the endometrium of the object to be detected, and obtains the position information of the identified endometrium according to a position of the target region of the endometrium in the three-dimensional volume data.

Herein, the preset characteristic may be a morphological characteristic, and the ultrasound imaging device performs binary segmentation on the three-dimensional volume data of the uterine region, and performs morphological operation processing on a result of the binary segmentation, so as to obtain the at least one candidate region of interest with a complete boundary. The morphological operation herein may be, for example, dilation processing or erosion processing of the result of the binary segmentation. The dilation processing may zoom in the edge of the result of the binary segmentation to a certain extent. The erosion processing can zoom out the result of the binary segmentation.

In the embodiment of the disclosure, in the volume data of the uterine region, echoes of the endometrium are significantly different from echoes of surrounding tissues, and as a woman's menstrual cycle changes, the morphology of the endometrium also shows periodic changes, with relatively obvious characteristics. Therefore, the detection of the endometrium can be implemented by using the method for detecting characteristics such as grayscale and/or morphology may be used to detect the endometrium.

In some embodiments of the disclosure, the specific implementation in which the ultrasound imaging device performs matching on the at least one candidate region of interest and the preset template region to identify a candidate region of interest with highest matching degree as a target region of the endometrium of the object to be detected may comprise: extracting a characteristic index of the at least one candidate region of interest, wherein the characteristic index comprises a shape characteristic, a texture characteristic, a boundary characteristic, or a grayscale distribution characteristic; calculating a relevancy between the at least one candidate region of interest and the preset template region based on the characteristic index; and using a candidate region of interest with highest relevancy and exceeding a preset threshold as the target region of the endometrium of the object to be detected.

It should be noted that the method for calculating a relevancy between the at least one candidate region of interest and the preset template region based on the characteristic index is not limited in the embodiment of the disclosure, and may involve characteristic matching, characteristic difference, etc.

In the embodiment of the disclosure, the preset threshold may be 90%, and the details thereof will be not limited in the embodiment of the disclosure.

Exemplarily, binary segmentation is performed on the three-dimensional volume data, and then, some necessary morphological operations are performed to obtain at least one candidate region of interest; then, for each candidate region of interest, the probability that each candidate region of interest is the endometrium is determined according to a shape characteristic; and a region with the highest probability is selected as a target region (i.e., with the highest matching degree). Specifically, the ultrasound imaging device can acquire, in advance, three-dimensional template data of the uterine region where the endometrium has been identified, obtain a preset template region of the endometrium according to the three-dimensional template data, and then perform matching on the at least one candidate region of interest and the preset template region, so as to identify a candidate region of interest with highest matching degree as a target region of the endometrium of the object to be detected.

That is to say, the ultrasound imaging device extracts shape characteristics from the three-dimensional volume data to obtain at least one candidate region of interest with different shape characteristics from the uterine region; the ultrasound imaging device compares the shape characteristics corresponding to the at least one candidate region of interest with shape characteristics of a preset template region to obtain at least one comparison result, wherein the at least one comparison result corresponds to the at least one candidate region of interest on a one-to-one basis; and the ultrasound imaging device identifies the candidate region of interest corresponding to the highest comparison result of the at least one comparison result as the endometrium (i.e., the target region), and acquires the position information of the identified endometrium (i.e., the position of the target region is in the three-dimensional volume data) from the three-dimensional data of the ultrasound image.

In the embodiment of the disclosure, the ultrasound imaging device may also implement the segmentation of the target region of the endometrium by using other grayscale detection and segmentation methods, such as OTSU threshold, Level Set, Graph Cut, and Snake, which are not limited in the embodiment of the disclosure.

In one embodiment of the disclosure, the endometrium may be detected based on a machine learning or deep learning method. When the machine learning or deep learning method is used, training is performed using a series of training samples by the ultrasound imaging device first; a preset positioning model is established; and then, the three-dimensional volume data of the uterine region is classified and regressed based on characteristics learned from the training, so as to obtain the position information of the endometrium in the three-dimensional volume data.

The ultrasound imaging device acquires the preset positioning model, which comprises three-dimensional positive sample data of the uterine region where the endometrium has been recognized, and calibration information of the recognized endometrium in the three-dimensional positive sample data; and the ultrasound imaging device identifies the endometrium from the three-dimensional volume data of the uterine region of the object to be detected based on the calibration information of the recognized endometrium in the preset locating model, so as to locate the position information of the identified endometrium.

In the embodiment of the disclosure, a method for locating and identifying a target region may be to detect or accurately segment a key anatomical structure (e.g., the endometrium) from the three-dimensional volume data by using the machine learning or deep learning method. For example, characteristics or patterns for distinguishing a target region (positive sample: endometrium region) and a non-target region (negative sample: background region) in the database can be learned first, and then key anatomical structures in other images are located and identified according to the learned characteristics or patterns.

It can be understood that the positive sample and the negative sample are used to train the preset positioning model here, such that a more comprehensive and accurate model may be obtained, and the accuracy of identification may be improved.

It should be noted that in the embodiment of the disclosure, the preset positioning model comprises three-dimensional positive sample data of the uterine region where the endometrium has been recognized, and calibration information of the recognized endometrium in the three-dimensional positive sample data, and the preset positioning model is obtained, by means of model training, using the machine learning or deep learning method. The three-dimensional positive sample data here refers to characteristic volume data containing the endometrium.

In some embodiment of the disclosure, the process of obtaining the preset positioning model by the ultrasound imaging device by means of model training is as follows: the ultrasound imaging device acquires three-dimensional training volume data of at least two objects to be trained, wherein the three-dimensional training volume data comprises at least the three-dimensional positive sample data of the uterine region where the endometrium has been recognized; the ultrasound imaging device calibrates, in the three-dimensional training volume data, the endometrium or an associated anatomical structure of the endometrium as calibration information of the endometrium in the three-dimensional training volume data; and the ultrasound imaging device performs model training by using a machine learning or deep learning method and based on the three-dimensional training volume data and the calibration information of the endometrium, so as to obtain the preset positioning model.

The preset positioning model represents a correlation between each piece of the three-dimensional volume data and the calibration information.

In the embodiment of the disclosure, the three-dimensional training volume data and the calibration information (i.e., a database) of the recognized endometrium are a plurality of pieces of volume data of the recognized endometrium and a result of calibration of the key anatomical structure. The result of calibration may be set according to actual task needs, may be a region of interest (ROI) box containing a target, or may be a mask for accurately segmenting the endometrium region, which is not limited in the embodiment of the disclosure.

In some embodiments of the disclosure, the ultrasound imaging device uses the calibration information of the recognized endometrium in the preset locating model to learn an image characteristic pattern of the endometrium by using a deep learning or machine learning method; and the ultrasound imaging device extracts, based on the endometrium image characteristic pattern, a target region containing the endometrium from the three-dimensional volume data of the uterine region of the object to be detected, and outputs position information of the target region in the three-dimensional volume data as the position information of the endometrium of the uterine region of the object to be detected.

That is to say, identifying the endometrium by the ultrasound imaging device may be divided into two steps: 1. acquiring a database, with the database comprising a plurality of pieces of three-dimensional training volume data and a result of calibration of the corresponding endometrium, wherein the result of calibration of the endometrium may be set according to actual task needs, may be a region of interest (ROI) box containing the endometrium, or may be a mask for accurately segmenting the endometrium; and 2. performing locating and identification, i.e., identifying and locating the region of interest in the ultrasound image by using a machine learning algorithm to learn characteristics or patterns capable of distinguishing a target region of the endometrium from a non-endometrium region in the database.

Optionally, the deep learning or machine learning method comprises: a sliding window-based method, a bounding-box method based on deep learning, and an end-to-end semantic segmentation network method based on deep learning; and by means of the above methods, the target region of the endometrium is calibrated, and a classifier is designed according to a result of calibration to classify and determine regions of interest. The specific selection of the methods is made according to actual situations, and is not specifically limited in the embodiment of the present application.

For example, the sliding window-based method may be: extracting characteristics from a region in a sliding window first, wherein the characteristic extraction method may involve principal components analysis (PCA), linear discriminant analysis (LDA), Harr characteristics, texture characteristics, etc., or a deep neural network may be used to extract characteristics, and then, performing matching on the extracted characteristics and a database, for classification by using a k-nearest neighbor (KNN) classification algorithm, a support vector machine (SVM), a random forest, a neural network and other discriminators, thereby determining whether the current sliding window is the target region of the endometrium and also acquiring a corresponding type thereof.

For example, the bounding-box method based on deep learning may be: performing characteristic learning and parameter regression on the constructed database by means of stacking of a basic convolutional layer and a fully connected layer; and for input three-dimensional volume data, the Bounding-Box of the target region of the corresponding endometrium can be directly obtained, by means of regression, through a network, and the type of a tissue structure in the target region of the endometrium is also acquired. Common networks comprise a regional-convolutional neural network (R-CNN), a fast region-convolutional neural network (Fast R-CNN), a Faster-RCNN, a single shot multibox detector (SSD), YOLO, etc.

For example, the end-to-end semantic segmentation network method based on deep learning may be: performing characteristic learning and parameter regression on the constructed database by means of stacking of a basic convolutional layer, upsampling, or a deconvolutional layer; and for input data, a bounding-box of the target region of the corresponding endometrium can be directly obtained, by means of regression, through a network, wherein any one of the upsampling or the deconvolutional layer is added to make the input and output have the same size, so as to directly obtain the target region of the endometrium in the input data and its corresponding type. Common networks comprise an FCN, a U-Net, a mask R-CNN, etc.

For example, it is also possible to first calibrate the target region of the endometrium by using the above three methods, and then design a classifier according to a result of calibration to classify and determine the target region of the endometrium. The method for classification and determination is: extracting characteristics from a target ROI or mask first, wherein the method for characteristic extraction may involve PCA, LDA, Haar characteristics, texture characteristics, etc., or a deep neural network may be used for characteristic extraction; and then, performing matching on the extracted characteristics and the database, for classification by using the KNN, the SVM, the random forest, the neural network and other discriminators.

In some embodiments of the disclosure, a series of profile image data may be extracted from the three-dimensional volume data, and then the endometrium is detected based on the profile image data.

The ultrasound imaging device acquires, from the three-dimensional volume data of the uterine region, sagittal image data in which the endometrium has been identified; the ultrasound imaging device determines a center point of the endometrium according to the sagittal image data; the ultrasound imaging device acquires, based on the center point, transverse image data that is orthogonal to the sagittal image data and is identified as comprising the endometrium; and the ultrasound imaging device obtains the position information of the endometrium based on positions of the transverse image data and sagittal image data that are identified as comprising the endometrium in the three-dimensional volume data of the uterine region.

It should be noted that the three-dimensional volume data in the embodiment of the disclosure is obtained by ultrasound scanning of the uterine region, then for a profile image formed based on the three-dimensional volume data, there may be a plurality of profile images comprising the endometrium. Therefore, the ultrasound imaging device detects the endometrium in some of the profile images in the three-dimensional volume data, which can also achieve the purpose of automatic imaging of the endometrium.

Exemplarily, when using the ultrasound imaging device to collect three-dimensional volume data, a doctor usually scans the uterine region with a sagittal plane as a starting section to obtain the three-dimensional volume data. Specifically, the method for detecting the endometrium based on a profile involves first acquiring, from the three-dimensional volume data, sagittal image data (side A) that is identified as comprising the endometrium, obtaining, from the sagittal image data, a center point of the endometrium in the sagittal image data, and determining, at the center point, section image data (side B) that is orthogonal to the sagittal data and comprises the endometrium. By means of the detection of the sides A and B, the position of the endometrium in the two orthogonal planes can be known. Although the position does not contain all target regions of the endometrium, it can also basically express a spatial position of the endometrium in the three-dimensional volume data, such that automatic imaging can be performed according to the position information of the endometrium.

It can be understood that in the embodiment of the disclosure, although the three-dimensional accurate detection of the endometrium is not directly performed in the three-dimensional volume data, it is only necessary to perform automatic detection on a few section images (e.g., a sagittal image and a transverse image) such that the approximate position information of the endometrium can be obtained, which greatly reduces the amount of calculation. Moreover, on the basis of acquiring sagittal image data, the ultrasound imaging device acquires transverse image data, such that the overturning that may occur during image acquisition is corrected.

It should be noted that in the embodiment of the disclosure, the method for detecting the endometrium based on profile image data, which is similar to the method for detecting a spatial position of the endometrium in the three-dimensional volume data, can also be implemented by using a method for detecting characteristics such as grayscale and/or morphology and a machine learning or deep learning algorithm, which will not be repeated herein.

In the embodiment of the disclosure, both the direct detection of the spatial position of the endometrium based on the three-dimensional volume data and the direct detection of the position of the endometrium in the profile image data aim to acquire the position of the endometrium in the three-dimensional volume data and to use same as a basis for follow-up imaging.

S105. Imaging is performed on the endometrium based on the three-dimensional volume data according to the position information of the endometrium, so as to obtain an image of the endometrium.

After the ultrasound imaging device has acquired the position information of the endometrium, the ultrasound device can perform imaging on the endometrium based on the three-dimensional volume data according to the position information of the endometrium, so as to obtain the image of the endometrium. When imaging is performed on the endometrium according to the position information of the endometrium in the three-dimensional volume data, the ultrasound imaging device can automatically acquire target volume data related to the endometrium from the three-dimensional volume data according to the position information, and then perform, in combination with a selected imaging method, image reconstruction and other processing on the target volume data, so as to obtain a corresponding ultrasound image.

It should be noted that after identifying the position information of the endometrium in the uterine region, i.e., after identifying a key anatomical structure in the uterine region, the ultrasound imaging device can implement the automatic imaging of the endometrium according to a position of the key anatomical structure in the three-dimensional volume data.

The ultrasound imaging device of the disclosure is a three-dimensional imaging system, which can implement the automatic imaging of the endometrium in three modes: VR imaging of the endometrium, CMPR imaging of the endometrium, and imaging of a standard section of the endometrium. The specific imaging method is not limited in the embodiment of the disclosure.

It should be noted that the ultrasound imaging device can extract a sagittal section image containing the endometrium from the three-dimensional volume data according to the position information of the endometrium, and then perform VR imaging and CMPR imaging based on the sagittal section image.

In some embodiments of the disclosure, the VR imaging performed by the ultrasound imaging device is rendering a region within a volume of interest (VOI) box, and the VOI box is usually a cuboid. During VR imaging of the endometrium, one flat plane of the cuboid may also be turned into a curved plane, and the curved plane can better conform to the curved structure of the endometrium.

During the VR imaging of the endometrium, a sagittal section image comprising the endometrium can be extracted from the three-dimensional volume data according to the position information of the endometrium; a preset drawing box is enabled, and adjustment processing is performed based on the preset drawing box, such that the preset drawing box covers the endometrium in the sagittal section image; and image drawing is performed for target three-dimensional volume data corresponding to the preset drawing box to obtain a three-dimensional image of the endometrium, wherein the target three-dimensional volume data is contained in the three-dimensional volume data of the uterine region.

In the embodiment of the disclosure, after the ultrasound imaging device has acquired the sagittal section image comprising the endometrium during VR imaging, the preset drawing box will be enabled, i.e., the preset drawing box will be displayed in the sagittal section image on the display of the ultrasound imaging device, and the adjustment processing is performed based on the preset drawing box, such that the preset drawing box covers the endometrium in the sagittal section image. In this case, VR image drawing is automatically performed on the target three-dimensional volume data of the three-dimensional volume data corresponding to the region within the preset drawing box.

It should be noted that the acquisition of VR images requires the adjustment to the orientation of the three-dimensional volume data (comprising endometrium volume data), or setting of the size and position of the VOI box, thereby achieving the purpose of just covering the endometrium in the sagittal section image by the preset drawing box. With regard to volume data of the endometrium, a user mainly focuses on the endometrium. Therefore, after the key anatomical structure, such as the endometrium, is detected, the orientation and size of the three-dimensional volume data can be automatically adjusted according to the position information of the endometrium, such that the VOI box can just enclose the endometrium region.

In the embodiment of the disclosure, when performing adjustment based on the preset drawing box, the ultrasound imaging device may adjust the size and position of the preset drawing box such that the preset drawing box covers the endometrium in the sagittal section image; or the ultrasound imaging device may adjust an orientation of the three-dimensional volume data of the uterine region according to the orientation of the preset drawing box in the sagittal section image, such that the preset drawing box covers the endometrium in the sagittal section image, or this may be implemented in other manners, which is not limited in the embodiment of the disclosure.

Specifically, the ultrasound imaging device can determine size and position of the endometrium in the sagittal section image according to the position information of the endometrium, and adjust size and position of the preset drawing box accordingly; and/or the ultrasound imaging device can determine an orientation of the endometrium in the three-dimensional volume data of the uterine region according to the position information of the endometrium, and adjust an orientation of the three-dimensional volume data of the uterine region according to the orientation of the preset drawing box in the sagittal section image.

In the embodiment of the disclosure, the preset drawing box is a volume of interest (VOI) box, and for three-dimensional stereoscopic imaging, VR imaging is to render the region within the preset drawing box to automatically form an image.

It should be noted that for the VOI, one flat plane of a cuboid may be turned into a curved plane, while the remaining five planes thereof is unchanged, wherein the curved plane may be used to observe a curved tissue structure. The purpose of setting the VOI box is to render only the region within the VOI box, but not to render a region beyond the VOI box, during stereoscopic rendering of the volume data, that is, a user can only see an image of imaging of the tissue within the VOI box through the VR image.

Further, in the embodiment of the disclosure, the curved plane of the VOI box overlaps with the curved lower edge of the endometrium as far as possible, such that the coronal image of the endometrium can be rendered.

Exemplarily, as shown in FIG. 4, in the sagittal section image of the three-dimensional volume data of the uterine region, after the position information of the endometrium has learned about, the preset drawing box 1 (VOI box) can be enabled. The preset drawing box 1 covers the endometrium, and the curved plane of VOI imaging overlaps with the lower edge of the endometrium as much as possible, such that VR imaging of the structure in the preset drawing box 1 is automatically performed to obtain the coronal image of the endometrium as shown in FIG. 5.

Here, coronal plane information of the endometrium may be displayed by using CMPR in addition to the three-dimensional reconstruction of a VR image.

It should be noted that CMPR imaging involves taking a trajectory curve in a certain section image of the three-dimensional volume data, and the trajectory curve cuts away the three-dimensional volume data to obtain a profile image of the curve, which can then be used to observe the curved tissue structure. Since the shape of the endometrium usually has a curved trajectory with a certain radian, directly taking a certain plane out of the three-dimensional volume data cannot completely display the coronal information of the endometrium. A CMPR section can well cover the trajectory of the entire endometrium so as to obtain a complete coronal image.

In the embodiment of the disclosure, a certain section image may be a sagittal section image or another section image, which is not limited in the embodiment of the disclosure.

Specifically, the ultrasound imaging device extracts a sagittal section image comprising the endometrium from the three-dimensional volume data according to the position information of the endometrium, and automatically generates a line of trajectory of the endometrium in the sagittal section image; and the ultrasound imaging device performs curved planar imaging of the endometrium for the three-dimensional volume data according to the line of trajectory, so as to obtain the image of the endometrium.

It should be noted that in the embodiment of the disclosure, the line of trajectory is a curve.

In the embodiment of the disclosure, after automatically identifying and obtaining the position information of the endometrium, the ultrasound imaging device automatically generates, in the sagittal section image and according to the position information of the endometrium, a CMPR line of trajectory that is sufficient to fit the endometrium, thereby implementing the automated CMPR imaging of the endometrium.

Due to a front-end scanning operation, in the acquired three-dimensional volume data of the uterine region, the endometrium may undergo a certain degree of twist. In this case, the orientation of the three-dimensional volume data needs to be adjusted such that the sagittal section image may display the endometrium as much as possible. Generally, an approximately elliptical image of the endometrium can be obtained in the transverse plane, and a preset transverse position of the endometrium in the transverse plane may be a horizontal position shown in the figure. The horizontal position may be, for example, a horizontal line shown by a dashed line in FIG. 10. If the endometrium is twisted, the image of the endometrium in the transverse plane may rotate at a certain angle, and a major axis of the elliptical image will no longer be the horizontal line shown in the figure, but will be inclined at a certain angle. As shown in FIG. 10, a white solid line represents the major axis of the transverse image of the endometrium, which is not aligned with the horizontal line shown by the dashed line, and this indicates that in this case, the endometrium has twisted at a certain angle. According to the above image characteristics of the endometrium in the transverse plane, the orientation of the three-dimensional volume data of the uterine region can be adjusted in the disclosure.

In some embodiments of the disclosure, the position information of the endometrium may comprise: a position of the endometrium in the sagittal plane and a position of the endometrium in the transverse plane. Then, the process of automatically generating a line of trajectory of the endometrium by the ultrasound imaging device in the sagittal section image may be: adjusting the orientation of the three-dimensional volume data until the position of the endometrium in the transverse plane is adjusted to conform to a preset transverse position, for example, the preset transverse position may be a horizontal position shown in FIG. 10; and determining the position of the endometrium in the sagittal plane based on the three-dimensional volume data after the orientation adjustment, and then automatically fitting the line of trajectory of the endometrium in the sagittal section image according to the position of the endometrium in the sagittal plane.

Exemplarily, the ultrasound imaging device respectively obtains positions of the endometrium in the sagittal plane and the transverse plane as sagittal position information and transverse position information, may rotate the position of the endometrium in the transverse plane to a horizontal state according to the position of the endometrium in the transverse plane (transverse position information), wherein the operation of rotation also involves adjusting the endometrium position of the endometrium in the sagittal plane, and then may fit a CMPR curve according to the adjusted position of the endometrium in the sagittal plane (sagittal position information), wherein the curve just passes through a central region of the endometrium. In this case, the CMPR image of the endometrium is obtained by means of imaging based on the CMPR curve.

It should be noted that in order to better display the endometrium, the three-dimensional volume data may also continue to be rotated or the CMPR image may be rotated, such that the endometrium is rotated to a vertical state to facilitate observation.

In the embodiment of the disclosure, in order to improve the contrast resolution and signal-to-noise ratio of the CMPR image, it can also be used in combination with a slice contrast view (SCV), wherein the SCV renders a region within a thickness range by the addition of thickness adjustment, such that the contrast resolution and signal-to-noise ratio of the image can be improved.

In some embodiments of the disclosure, the ultrasound imaging device extracts a sagittal section image comprising the endometrium from the three-dimensional volume data according to the position information of the endometrium, and automatically generates a line of trajectory of the endometrium in the sagittal section image; the ultrasound imaging device acquires edge information of the endometrium in the sagittal section image according to the position information of the endometrium; the ultrasound imaging device determines an image drawing region according to the edge information and the line of trajectory; and the ultrasound imaging device performs curved planar imaging of the endometrium for target three-dimensional volume data corresponding to the image drawing region, so as to obtain an image of the endometrium that reflects a thickness of the endometrium.

It should be noted that the ultrasound imaging device extracts the sagittal section image comprising the endometrium from the three-dimensional volume data according to the position information of the endometrium, and automatically generates, in the sagittal section image, a line of trajectory of the endometrium, which is a single curve and cannot represent the thickness of the endometrium. In this case, edge information of the endometrium in the sagittal section image can be acquired according to the position information of the endometrium. In this way, a region with a certain thickness between the line of trajectory and the edge information is determined as the image drawing region, and the image of the endometrium that reflects the thickness of the endometrium is obtained only by performing curved planar imaging of the endometrium on the target three-dimensional volume data corresponding to the image drawing region. Since the image of the endometrium with an endometrium thickness is obtained in this way, the resolution of the image is improved.

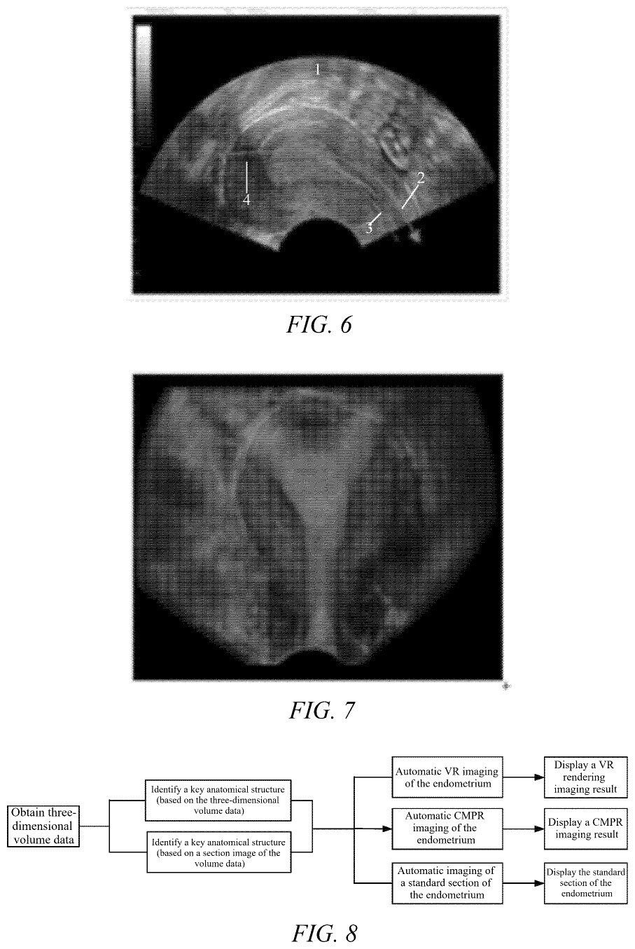

Exemplarily, as shown in FIG. 6, the ultrasound imaging device extracts a sagittal section image 1 comprising the endometrium from the three-dimensional volume data according to the position information of the endometrium, and automatically generates a line of trajectory 2 of the endometrium in the sagittal section image 1; the ultrasound imaging device acquires edge information 3 of the endometrium in the sagittal section image 1 according to the position information of the endometrium; the ultrasound imaging device determines an image drawing region 4 according to the edge information 3 and the line of trajectory 2; and the ultrasound imaging device performs curved planar imaging of the endometrium for target three-dimensional volume data corresponding to the image drawing region 4, so as to obtain an image of the endometrium that reflects a thickness of the endometrium (as shown in FIG. 7).

In the embodiment of the disclosure, the ultrasound imaging device can also obtain a section image of the endometrium by means of two-dimensional imaging. Based on the position information of the endometrium detected in the three-dimensional volume data, a standard section of the endometrium can also be directly obtained by means of planar imaging.

For example, the ultrasound imaging device fits a coronal plane of the endometrium according to the position information of the endometrium; the ultrasound imaging device acquires a grayscale image corresponding to the coronal plane of the endometrium from the three-dimensional volume data; and the ultrasound imaging device uses the grayscale image as the standard section image of the endometrium. The standard section image here is a coronal section image of the endometrium.

It should be noted that the endometrium usually is of a curved structure, which may be better expressed by means of VR imaging or CMPR. However, as an approximation, the coronal plane of the endometrium may also be directly displayed in a plane.

That is to say, after detecting the position information of the endometrium in the three-dimensional volume data, the ultrasound imaging device can fit the coronal plane of the endometrium, such that the plane passes through the endometrium region and can maximize the display of the endometrium (the endometrium is a sheet-shaped object of a certain thickness, and the coronal plane is a central plane of the sheet-shaped object). An equation of the plane can be obtained by means of equation solving or least squares estimation fitting. After the equation of the plane is obtained, a grayscale image corresponding to the plane can be acquired from the three-dimensional volume data to obtain a standard section of the endometrium. In addition, based on the position information of the endometrium in the three-dimensional volume data, rotation and correction can be performed on an angular deviation thereof to finally obtain a section image (two-dimensional plane) of the endometrium.

In the embodiment of the disclosure, all of the above imaging methods can be used to generate an image of the endometrium, and they can be used independently or in combination, which is not limited in the embodiment of the disclosure.

It should be noted that where the image quality is poor and there is a deviation in the position of the anatomical structure of the endometrium that is detected by using an algorithm, a user can also perform modification operations, such as moving, zooming, deletion and re-calibration, on a detected VOI region or CMPR curve in the section by means of a keyboard, a mouse and other tools, so as to implement semi-automatic VOI imaging or CMPR curved planar imaging; and for the imaging of the standard section of the endometrium, the user can also adjust the section through a knob, which is not limited in the embodiment of the disclosure.

S106. The image of the identified endometrium is displayed.

After the ultrasound imaging device has acquired the images of the endometrium, the ultrasound imaging device displays the images of the endometrium on a display, and these images of the endometrium are stored in a memory.

In the embodiment of the disclosure, during the automatic VR imaging of the endometrium, the ultrasound imaging device obtains a VR image of the endometrium by using a three-dimensional rendering algorithm, such as ray tracing, and displays the image on the display.

In the embodiment of the disclosure, during the automatic CMPR imaging of the endometrium, the ultrasound imaging device acquires a CMPR image of the endometrium and displays the image on the display.

In the embodiment of the disclosure, the ultrasound imaging device obtains a standard section image of the endometrium by means of automatic imaging based on the standard section of the endometrium.

In some embodiments, a certain workflow may be set to integrate functions corresponding to different imaging modes into the workflow for a doctor to freely choose, and images corresponding to the selected functions are displayed on the display.

Exemplarily, as shown in FIG. 8, the ultrasound imaging device obtains three-dimensional volume data of the uterine region through ultrasonic waves and detects a key anatomical structure, which is specifically as follows: performing characteristic identification based on the three-dimensional volume data to identify the key anatomical structure (endometrium), i.e., the region of interest, or identifying the key anatomical structure, i.e., the region of interest, based on a profile image of the three-dimensional volume data. After the endometrium is identified, at least one of automatic VR imaging of the endometrium, automatic CMPR imaging of the endometrium, and automatic imaging of a standard section of the endometrium is used to obtain an image of the endometrium (automatic imaging of the endometrium), and display an imaging result. For example, a VR rendering imaging result, a CMPR imaging result, or the standard section of the endometrium is displayed.

It can be understood that the ultrasound imaging device can identify the endometrium by identifying the three-dimensional volume data of the uterine region of an object to be detected, so as to obtain the position information of the endometrium, and thus obtain the section image of the endometrium by means of automatic imaging. The position of the endometrium that is found in this way is accurate, which improves the accuracy of ultrasound imaging, and the automatic imaging further improves the intelligence of imaging of an ultrasound image.

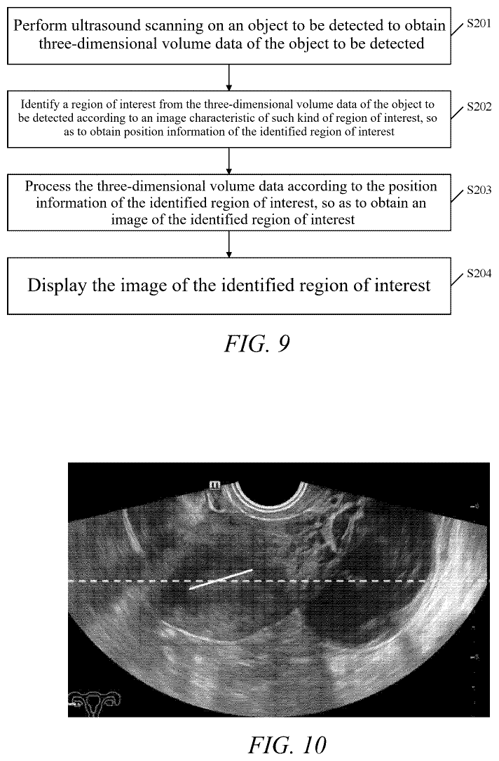

Based on the above implementation, taking the key anatomical structure as the region of interest as an example, a method for ultrasound imaging of a region of interest is provided. As shown in FIG. 9, the method may comprise:

S201. performing ultrasound scanning on an object to be detected, so as to obtain three-dimensional volume data of the object to be detected;

S202. identifying a region of interest from the three-dimensional volume data of the object to be detected according to an image characteristic of such kind of region of interest, so as to obtain position information of the identified region of interest;

S203. processing the three-dimensional volume data according to the position information of the identified region of interest, so as to obtain an image of the identified region of interest; and

S204. displaying the image of the identified region of interest.

In the embodiment of the disclosure, the identifying the region of interest from the three-dimensional volume data of the object to be detected by the ultrasound imaging device according to an image characteristic of such kind of region of interest, so as to obtain position information of the identified region of interest, comprises the methods as follows.

(1) A preset characteristic is extracted from the three-dimensional volume data to obtain at least one candidate region of interest; and matching is performed on the at least one candidate region of interest and a preset template region to identify a region of interest with highest matching degree, so as to obtain the position information of the identified region of interest.

(2) The three-dimensional volume data is processed based on a preset positioning model to identify the region of interest in the object to be detected, so as to locate the position information of the identified region of interest, wherein the preset locating model represents a correlation between the three-dimensional volume data and the region of interest.

(3) Sagittal image data of the region of interest is acquired from the three-dimensional volume data; a center point of the region of interest is determined according to the sagittal image data; transverse image data that is orthogonal to the sagittal image data is acquired based on the center point; and the region of interest is identified based on the transverse image data and the sagittal image data, so as to obtain the position information of the identified region of interest.

In the embodiment of the disclosure, before the processing the three-dimensional volume data based on a preset positioning model to identify the region of interest in the object to be detected, so as to locate the position information of the positioning region of interest, the preset positioning model needs to be acquired first. The preset locating model can be constructed in advance, and the constructed preset positioning model is invoked during the imaging process. The process of constructing the preset positioning model may comprise: acquiring three-dimensional training volume data and regions of interest of at least two objects to be trained; and training a training model by using a preset machine learning algorithm and based on the three-dimensional training volume data and the regions of interest, so as to obtain the preset locating model.

In the embodiment of the disclosure, processing the three-dimensional volume data by the ultrasound imaging device according to the position information of the identified region of interest so as to obtain a section image of the identified region of interest comprises the methods as follows.

(1) A preset drawing box is acquired; a target region of interest corresponding to the position information of the identified region of interest is covered by the preset drawing box; and image drawing is performed for target three-dimensional volume data corresponding to the preset drawing box to obtain a three-dimensional image of the identified region of interest, wherein the target three-dimensional volume data is contained in the three-dimensional volume data.

(2) A line of trajectory of the region of interest is generated according to the position information of the identified region of interest; and image drawing of the identified region of interest is performed for the three-dimensional volume data according to the line of trajectory, so as to obtain the image of the identified region of interest.

(3) Edge information of the identified region of interest is acquired; an image drawing region is determined according to the edge information and the line of trajectory; and image drawing of the identified region of interest is performed for the three-dimensional volume data according to the image drawing region, so as to obtain the three-dimensional image of the identified region of interest.

(4) A coronal plane of the identified region of interest is fitted according to the position information of the identified region of interest; a grayscale image corresponding to the coronal plane of the region of interest is acquired from the three-dimensional volume data; and the grayscale image is used as a standard section image of the identified region of interest.

It should be noted that the position information of the region of interest may comprise: sagittal position information of the identified region of interest and transverse position information of the identified region of interest. The process of generating a line of trajectory of the identified region of interest according to the position information of the identified region of interest is: adjusting the transverse position information of the identified region of interest to the same horizontal plane as the sagittal position information of the identified region of interest to obtain rotated-transverse position information of the identified region of interest; and fitting the line of trajectory of the identified region of interest according to the rotated-transverse position information and the sagittal position information.

It should be noted that the principle and implementation method of the implementation process of S201 to S204 are consistent with the implementation principle of S101 to S106 above, and will not be repeated herein.

An embodiment of the disclosure provides an ultrasound imaging device. As shown in FIG. 1, the ultrasound imaging device comprises:
a probe 100;
a transmitting circuit 101 configured to excite the probe 100 to transmit an ultrasonic wave to an object to be detected;
a transmitting/receiving selection switch 102;
a receiving circuit 103 configured to receive, by the probe 100, an ultrasonic echo reflected from the object to be detected, so as to obtain an ultrasonic echo signal or data;
a beam synthesis circuit 104 configured to perform beam synthesis processing on the ultrasonic echo signal data to obtain an ultrasonic echo signal or data which have been subjected to beam synthesis;
a processor 105 configured to: process the ultrasonic echo signal which have been subjected to beam synthesis, so as to obtain three-dimensional volume data of the uterine region of the object to be detected; identify an endometrium from the three-dimensional volume data of the uterine region according to an image endometrium characteristic in the uterine region, so as to obtain position information of the identified endometrium; and perform imaging on the identified endometrium based on the three-dimensional volume data according to the position information of the identified endometrium, so as to obtain an image of the identified endometrium; and
a display 106 configured to display the image of the identified endometrium.

In some embodiments of the disclosure, the processor 105 may be configured to identify the endometrium from the three-dimensional volume data of the uterine region according to a difference between the endometrium image characteristics and a basal tissue image characteristics of an uterus in an uterine region, or according to a periodically changing morphological characteristic of an endometrium in the uterine region, so as to obtain the position information of the identified endometrium.

In some embodiments of the disclosure, the processor 105 may be configured to: extract a preset characteristic from the three-dimensional volume data of the uterine region to obtain at least one candidate region of interest; acquire three-dimensional template data of the uterine region where an endometrium has been recognized, and obtain a preset template region of the recognized endometrium according to the three-dimensional template data; and perform matching on the at least one candidate region of interest and the preset template region to identify a candidate region of interest with highest matching degree as a target region of the endometrium of the object to be detected, and obtain the position information of the identified endometrium according to a position of the target region of the endometrium in the three-dimensional volume data.

In some embodiments of the disclosure, the processor 105 may be further configured to: extract a characteristic index of the at least one candidate region of interest, wherein the characteristic index comprises a shape characteristic, a texture characteristic, a boundary characteristic, or a grayscale distribution characteristic; calculate a relevancy between the at least one candidate region of interest and the preset template region based on the characteristic index; and use a candidate region of interest with highest relevancy t and exceeding a preset threshold as the target region of the endometrium of the object to be detected.

In some embodiments of the disclosure, the processor 105 may be configured to perform image segmentation on the three-dimensional volume data of the uterine region, and perform morphological operation processing on a result of the image segmentation to obtain the at least one candidate region of interest with a complete boundary.

In some embodiments of the disclosure, the processor 105 may be configured to: acquire

a preset positioning model, wherein the preset positioning model comprises three-dimensional positive sample data of an uterine region where an endometrium has been recognized, and calibration information of the recognized endometrium in the three-dimensional positive sample data; and identify the endometrium from the three-dimensional volume data of the uterine region of the object to be detected based on the calibration information of the recognized endometrium in the preset locating model, so as to locate the position information of the identified endometrium.

In some embodiments of the disclosure, the processor 105 may be further configured to: use the calibration information of the endometrium in the preset positioning model to learn an endometrium image characteristic pattern by using a deep learning or machine learning method; and extract, based on the endometrium image characteristic pattern, a target region containing the endometrium from the three-dimensional volume data of the uterine region of the object to be detected, and output position information of the target region in the three-dimensional volume data as the position information of the endometrium of the uterine region of the object to be detected.

In some embodiments of the disclosure, the processor 105 may be further configured to: acquire three-dimensional training volume data of at least two objects to be trained, wherein the three-dimensional training volume data comprises at least the three-dimensional template data with the recognized endometrium of the uterine region; calibrate, in the three-dimensional training volume data, the recognized endometrium or an associated anatomical structure of the recognized endometrium as calibration information of the recognized endometrium in the three-dimensional training volume data; and train a training model by using a machine learning or deep learning method and based on the three-dimensional training volume data and the calibration information of the recognized endometrium, so as to obtain the preset positioning model.

In some embodiments of the disclosure, the processor 105 may be configured to: acquire, from the three-dimensional volume data of the uterine region, sagittal image data in which the endometrium has been identified; determine a center point of the endometrium according to the sagittal image data; acquire, based on the center point, transverse image data that is orthogonal to the sagittal image data and is identified as comprising the endometrium; and obtain the position information of the endometrium based on positions of the transverse image data and position information of the sagittal image data.

In some embodiments of the disclosure, the processor 105 may be configured to: extract a sagittal section image comprising the identified endometrium from the three-dimensional volume data according to the position information of the identified endometrium; enable and adjust a preset drawing box, such that the preset drawing box covers the identified endometrium in the sagittal section image; and perform image drawing for target three-dimensional volume data corresponding to the preset drawing box to obtain a three-dimensional section image of the identified endometrium, wherein the target three-dimensional volume data is contained within the three-dimensional volume data of the uterine region.

In some embodiments of the disclosure, the processor 105 may be further configured to: determine size and position of the endometrium in the sagittal section image according to the position information of the identified endometrium, and adjust size and position of the preset drawing box accordingly; or determine an orientation of the identified endometrium in the three-dimensional volume data of the uterine region according to the position information of the identified endometrium, and adjust an orientation of the three-dimensional volume data of the uterine region according to an orientation of the preset drawing box in the sagittal section image.

In some embodiments of the disclosure, the processor 105 may be configured to: extract a sagittal section image comprising the identified endometrium from the three-dimensional volume data according to the position information of the identified endometrium, and automatically generate a line of trajectory of the identified endometrium in the sagittal section image; and perform curved planar imaging of the identified endometrium for the three-dimensional volume data according to the line of trajectory, so as to obtain the image of the identified endometrium.

In some embodiments of the disclosure, the position information of the endometrium comprises: sagittal position information and transverse position information.

The processor 105 may be further configured to: adjust an orientation of the three-dimensional volume data of the uterine region until the position of the identified endometrium in the transverse plane conforms to a preset transverse position, wherein the preset transverse position may be a horizontal position shown in FIG. 10, for example; and determine the position of the identified endometrium in the sagittal plane based on the three-dimensional volume data with the adjusted orientation of the uterine region, and then automatically fit the line of trajectory of the identified endometrium in the sagittal section image according to the position of the identified endometrium in the sagittal plane.

In some embodiments of the disclosure, the processor 105 may be further configured to: after automatically generating the line of trajectory of the identified endometrium in the sagittal section image, acquire edge information of the identified endometrium in the sagittal section image according to the position information of the identified endometrium; determine an image drawing region according to the edge information and the line of trajectory; and perform curved planar imaging of the identified endometrium for target three-dimensional volume data corresponding to the image drawing region, so as to obtain a three-dimensional image of the identified endometrium that reflects a thickness of the endometrium.

In some embodiments of the disclosure, the processor 105 may be configured to: fit a coronal plane of the identified endometrium according to the position information of the identified endometrium; acquire a grayscale image corresponding to the coronal plane of the identified endometrium from the three-dimensional volume data; and use the grayscale image as a standard section image of the identified endometrium.

It can be understood that the ultrasound imaging device can identify the endometrium by identifying the three-dimensional volume data of the uterine region of the object to be detected, so as to obtain the position information of the endometrium, thereby omitting cumbersome operations of continuously and manually locating the endometrium by a user, facilitating quick identification of the endometrium by the user, and improving the overall working efficiency. The ultrasound imaging device may further perform automatic imaging according to the position information of the endometrium to obtain an image of the endometrium, and since the automatically identified position of the endometrium is accurate, the accuracy of ultrasound imaging is improved, and the automatic imaging may also improve the intelligence of the imaging of an ultrasound image.

An embodiment of the disclosure provides a computer-readable storage medium storing an ultrasound imaging program, wherein the ultrasound imaging program may be executed by a processor to implement the above ultrasound imaging method.

The computer-readable storage medium may be a volatile memory, such as a random-access memory (RAM), or may be a non-volatile memory, such as a read-only memory (ROM), a flash memory, a hard disk drive (HDD) or a solid-state drive (SSD); alternatively, the computer-readable storage medium may be a respective device comprising one or any combination of the above memories, for example, a mobile phone, a computer, a tablet device, a personal digital assistant, etc.

What is claimed is:

1. An ultrasound imaging method, comprising:
transmitting an ultrasonic wave to a uterine region of an object to be detected for volume scanning;
receiving an ultrasonic echo reflected from the uterine region of the object to be detected, and acquiring an ultrasonic echo signal based on the ultrasonic echo;
processing the ultrasonic echo signal to obtain three-dimensional volume data of the uterine region of the object to be detected;
identifying an endometrium from the three-dimensional volume data of the uterine region according to an endometrium image characteristic, so as to obtain position information of the identified endometrium, wherein the position information represents a position of the identified endometrium in the three-dimensional volume data;
performing imaging on the identified endometrium based on the three-dimensional volume data according to the position information of the identified endometrium, so as to obtain an image of the identified endometrium, wherein the obtained image of the identified endometrium comprises a Computed Multiplanar Reformation (CMPR) imaging of the identified endometrium, the CMPR imaging of the identified endometrium involves taking a trajectory curve of the endometrium in a section image of the three-dimensional volume data of the uterine region, and the trajectory curve of the endometrium cuts away the three-dimensional volume data of the uterine region to obtain a profile image of the trajectory curve of the endometrium; and
displaying the image of the identified endometrium.

2. The method of claim 1, wherein identifying an endometrium from the three-dimensional volume data of the uterine region according to an endometrium image characteristic, so as to obtain position information of the identified endometrium comprises:
identifying the endometrium from the three-dimensional volume data of the uterine region according to a difference between the endometrium image characteristic and a basal tissue image characteristic of a uterus, or according to a periodically changing morphological characteristic of an endometrium in a uterine region, so as to obtain the position information of the identified endometrium.

3. The method of claim 1, wherein identifying an endometrium from the three-dimensional volume data of the uterine region according to an endometrium image characteristic, so as to obtain position information of the identified endometrium comprises:
extracting a preset characteristic from the three-dimensional volume data of the uterine region to obtain at least one candidate region of interest;
acquiring three-dimensional template data of a uterine region where an endometrium has been recognized, and obtaining a preset template region of the recognized endometrium according to the three-dimensional template data; and
matching the at least one candidate region of interest with the preset template region to identify a candidate region of interest with highest matching degree as a target region of the endometrium of the object to be detected, and obtaining the position information of the identified endometrium according to a position of the target region of the endometrium in the three-dimensional volume data.

4. The method of claim 3, wherein matching the at least one candidate region of interest with the preset template region to identify a candidate region of interest with highest matching degree as a target region of the endometrium of the object to be detected comprises:
extracting a characteristic index of the at least one candidate region of interest, wherein the characteristic index comprises a shape characteristic index, a texture characteristic index, a boundary characteristic index, or a grayscale distribution characteristic index;
calculating a relevancy between the at least one candidate region of interest and the preset template region based on the characteristic index; and
using a candidate region of interest with highest relevancy and exceeding a preset threshold as the target region of the endometrium of the object to be detected.

5. The method of claim 3, wherein extracting a preset characteristic from the three-dimensional volume data of the uterine region to obtain at least one candidate region of interest comprises:
performing image segmentation on the three-dimensional volume data of the uterine region, and performing morphological operation processing on a result of the image segmentation to obtain the at least one candidate region of interest with a complete boundary.

6. The method of claim 1, wherein identifying an endometrium from the three-dimensional volume data of the uterine region according to an endometrium image characteristic, so as to obtain position information of the identified endometrium comprises:
acquiring a preset positioning model, wherein the preset positioning model comprises three-dimensional positive sample data of a uterine region where an endometrium has been recognized, and calibration information of the recognized endometrium in the three-dimensional positive sample data; and
identifying the endometrium from the three-dimensional volume data of the uterine region of the object to be detected based on the calibration information of the recognized endometrium in the preset positioning model, so as to locate the position information of the identified endometrium.

7. The method of claim 6, wherein identifying the endometrium from the three-dimensional volume data of the uterine region of the object to be detected based on the calibration information of the recognized endometrium in the preset positioning model, so as to locate the position information of the identified endometrium comprises:
acquiring, based on the calibration information of the recognized endometrium in the preset positioning model, an endometrium image characteristic pattern by using a deep learning method; and
extracting, based on the endometrium image characteristic pattern, a target region containing the endometrium from the three-dimensional volume data of the uterine region of the object to be detected, and outputting position information of the target region in the three-dimensional volume data as the position information of the endometrium of the uterine region of the object to be detected.

8. The method of claim 6, wherein acquiring a preset positioning model comprises:
acquiring three-dimensional training volume data of at least two objects to be trained, wherein the three-dimensional training volume data comprises at least the three-dimensional positive sample data with the recognized endometrium of the uterine region;
calibrating, in the three-dimensional training volume data, the recognized endometrium or an associated anatomical structure of the recognized endometrium as calibration information of the recognized endometrium in the three-dimensional training volume data; and
performing model training by using a machine learning or deep learning method, based on the three-dimensional training volume data and the calibration information of the recognized endometrium, so as to obtain the preset positioning model.

9. The method of claim 1, wherein identifying an endometrium from the three-dimensional volume data of the uterine region according to an endometrium image characteristic, so as to obtain position information of the identified endometrium comprises:
acquiring, from the three-dimensional volume data of the uterine region, sagittal image data in which the endometrium has been identified;
determining a center point of the endometrium according to the sagittal image data;
acquiring, based on the center point, transverse image data that is orthogonal to the sagittal image data and is identified as comprising the endometrium; and
obtaining the position information of the endometrium based on position information of the transverse image data and position information of the sagittal image data.

10. The method of claim 1, wherein performing imaging on the identified endometrium based on the three-dimensional volume data according to the position information of the identified endometrium, so as to obtain an image of the identified endometrium comprises:
extracting a sagittal section image comprising the identified endometrium from the three-dimensional volume data according to the position information of the identified endometrium, and automatically generating a line of trajectory of the identified endometrium in the sagittal section image; and
performing curved planar imaging of the identified endometrium for the three-dimensional volume data according to the line of trajectory, so as to obtain the image of the identified endometrium.

11. The method of claim 10, wherein the position information of the identified endometrium comprises: a position of the identified endometrium in a sagittal plane and a position of the identified endometrium in a transverse plane; and automatically generating a line of trajectory of the identified endometrium in the sagittal section image comprises:
adjusting an orientation of the three-dimensional volume data of the uterine region until the position of the identified endometrium in the transverse plane conforms to a preset position of the identified endometrium in the transverse plane; and
determining the position of the identified endometrium in the sagittal plane based on the three-dimensional volume data with the adjusted orientation of the uterine region, and automatically fitting the line of trajectory of the identified endometrium in the sagittal section image according to the position of the identified endometrium in the sagittal plane.

12. The method of claim 10, wherein after automatically generating the line of trajectory of the identified endometrium in the sagittal section image, the method further comprises:
acquiring edge information of the identified endometrium in the sagittal section image according to the position information of the identified endometrium;
determining an image drawing region according to the edge information and the line of trajectory; and
performing curved planar imaging of the identified endometrium for target three-dimensional volume data corresponding to the image drawing region, so as to obtain an image of the identified endometrium that reflects a thickness of the identified endometrium.

13. An ultrasound imaging device, comprising:
a probe;
a transmitting circuit configured to excite the probe to transmit an ultrasonic wave to an object to be detected for volume scanning;
a transmitting or receiving selection switch;
a receiving circuit configured to receive, by the probe, an ultrasonic echo reflected from the object to be detected, so as to obtain an ultrasonic echo signal or data;
a beam synthesis circuit configured to perform beam synthesis processing on the ultrasonic echo signal or data to obtain an ultrasonic echo signal or data which has been subjected to beam synthesis;
a processor configured to: process the ultrasonic echo signal which have been subjected to beam synthesis, so as to obtain three-dimensional volume data of an uterine region of the object to be detected; identify an endometrium from the three-dimensional volume data of the uterine region according to an endometrium image characteristic, so as to obtain position information of the identified endometrium, wherein the position information represents a position of the identified endometrium in the three-dimensional volume data; and perform imaging on the identified endometrium based on the three-dimensional volume data according to the position information of the identified endometrium, so as to obtain an image of the identified endometrium, wherein the obtained image of the identified endometrium comprises a Computed Multiplanar Reformation (CMPR) imaging of the identified endometrium, the CMPR imaging of the identified endometrium involves taking a trajectory curve of the endometrium in a section image of the three-dimensional volume data of the uterine region, and the trajectory curve of the endometrium cuts away the three-dimensional volume data of the uterine region to obtain a profile image of the trajectory curve of the endometrium; and
a display configured to display the image of the identified endometrium.

14. The device of claim 13, wherein the processor is configured to:
identify the endometrium from the three-dimensional volume data of the uterine region according to a difference between the endometrium image characteristic and a basal tissue image characteristic of a uterus in a uterine region, or according to a periodically changing morphological characteristic of an endometrium in a uterine region, so as to obtain the position information of the identified endometrium.

15. The device of claim 13, wherein the processor is configured to:
extract a preset characteristic from the three-dimensional volume data of the uterine region to obtain at least one candidate region of interest;
acquire three-dimensional template data of a uterine region where an endometrium has been recognized, and obtain a preset template region of the recognized endometrium according to the three-dimensional template data; and
match the at least one candidate region of interest with the preset template region to identify a candidate region of interest with a highest matching degree as a target region of the endometrium of the object to be detected, and obtain the position information of the identified endometrium according to a position of the target region of the endometrium in the three-dimensional volume data.

16. The device of claim 15, wherein the processor is further configured to:
extract a characteristic index of the at least one candidate region of interest, wherein the characteristic index comprises a shape characteristic index, a texture characteristic index, a boundary characteristic index, or a grayscale distribution characteristic index;
calculate a relevancy between the at least one candidate region of interest and the preset template region based on the characteristic index; and
use a candidate region of interest with highest relevancy and exceeding a preset threshold as the target region of the endometrium of the object to be detected.

17. The device of claim 15, wherein the processor is configured to:
perform image segmentation on the three-dimensional volume data of the uterine region, and perform morphological operation processing on a result of the image segmentation to obtain the at least one candidate region of interest with a complete boundary.

18. The device of claim 13, wherein the processor is configured to:
acquire a preset positioning model, wherein the preset positioning model comprises three-dimensional template data of a uterine region where an endometrium has been recognized, and calibration information of the recognized endometrium in the three-dimensional template data; and
identify the endometrium from the three-dimensional volume data of the uterine region of the object to be detected based on the calibration information of the recognized endometrium in the preset positioning model, so as to locate the position information of the identified endometrium.

19. The device of claim 18, wherein the processor is further configured to:
acquire, based on the calibration information of the recognized endometrium in the preset positioning model, an endometrium image characteristic pattern by using a deep learning or machine learning method; and
extract, based on the endometrium image characteristic pattern, a target region containing the endometrium from the three-dimensional volume data of the uterine region of the object to be detected, and output position information of the target region in the three-dimensional volume data as the position information of the endometrium of the uterine region of the object to be detected.

20. A non-transitory computer-readable storage medium storing instructions which, when executed by one or more processors, cause the one or more processors to perform operations comprising:
transmitting an ultrasonic wave to a uterine region of an object to be detected for volume scanning;
receiving an ultrasonic echo reflected from the uterine region of the object to be detected, and acquiring an ultrasonic echo signal based on the ultrasonic echo;
processing the ultrasonic echo signal to obtain three-dimensional volume data of the uterine region of the object to be detected;
identifying an endometrium from the three-dimensional volume data of the uterine region according to an endometrium image characteristic, so as to obtain position information of the identified endometrium, wherein the position information represents a position of the identified endometrium in the three-dimensional volume data;
performing imaging on the identified endometrium based on the three-dimensional volume data according to the position information of the identified endometrium, so as to obtain an image of the identified endometrium, wherein the obtained image of the identified endometrium comprises a Computed Multiplanar Reformation (CMPR) imaging of the identified endometrium, the CMPR imaging of the identified endometrium involves taking a trajectory curve of the endometrium in a section image of the three-dimensional volume data of the uterine region, and the trajectory curve of the endometrium cuts away the three-dimensional volume data of the uterine region to obtain a profile image of the trajectory curve of the endometrium; and
displaying the image of the identified endometrium.
